(19) United States
(12) Patent Application Publication
(10) Pub. No.: US 2012/0265282 A1
(43) Pub. Date: Oct. 18, 2012

(54) CAROTID SHEATH WITH THIN-WALLED SHAFT AND VARIABLE STIFFNESS ALONG ITS LENGTH

(75) Inventors: ROBERT E. FISCHELL, DAYTON, MD (US); TIM A. FISCHELL, KALAMAZOO, MI (US)

(73) Assignee: FISCHELL INNOVATIONS LLC, FAIR HAVEN, NJ (US)

(21) Appl. No.: 13/085,951

(22) Filed: Apr. 13, 2011

(51) Int. Cl. A61F 2/84

(52) U.S. Cl. 623/1.11

(57) ABSTRACT

A sheath to access a patient's vascular system where a portion of the length of the sheath is the proximal portion which has stiffer bending characteristics when taken with respect to a shorter distal section of the sheath which has increased flexibility with regard to bending characteristics.

### FIELD OF USE

This invention is in the field of devices to assist in the placement of catheters through the skin to treat carotid artery obstructive disease.

### BACKGROUND OF THE INVENTION

At the present time, physicians often treat carotid artery obstructive disease with the placement of a stent. This stent is typically placed in the internal carotid artery, in the common carotid artery, or spanning both arteries with the distal portion of the stent in the internal carotid artery and the proximal portion of the stent in the distal common carotid artery. The start of this procedure necessitates the placement of either a long sheath or a guiding catheter into the common carotid artery proximal to the carotid stenosis to be treated. The placement of such a sheath or guiding catheter can often be extremely challenging due to the tortuous course for access from the aortic arch into the common carotid artery. This is particularly an issue when accessing the right common carotid artery, which typically arises as a proximal branch from the inominate artery. Many different “tricks” are used to try to place relatively stiff sheaths and guiding catheters into the carotid circulation. One such “trick” is to have the sheath track over a “super-stiff” guidewire. Even with the best of equipment, it can be technically challenging, or even impossible to access the common carotid artery in order to stent a stenosis at that location when using any existing carotid sheath that has a uniform flexibility along its entire length. If stenting is not possible, then the more demanding and potentially life threatening procedure of a surgical endarterectomy would be required. Therefore, it is urgently needed to have for the interventional cardiologist an improved carotid sheath that allows for more successful guidance through the tortuous vascular anatomy that is encountered when attempting to stent a stenosis in any carotid artery.

Another problem with current approach for carotid stenting is that it requires the placement of a relatively large sheath (typically 8 French) or a thick walled 7 French carotid sheath system to deliver the relatively high profile carotid stent delivery catheter. The use of these larger diameter sheaths can lead to vascular access bleeding after the sheath has been removed. In general, there is a relationship between the outer diameter of the inserted sheath and the risk of bleeding complications. Thus, sheaths with thinner walls would have a smaller outside diameter and that would decrease the size of the hole at the vascular entry site and doing that would reduce bleeding complications.

### SUMMARY OF THE INVENTION

A sheath diameter is typically expressed in FR (read “French”) which is the diameter of the sheath in millimeters divided by three. So a 6 FR sheath has a diameter of 2 mm. Using the currently available technology for carotid stenting, it is typical to use a relatively small sized (5 FR or 6 FR) sheath and diagnostic catheter (e.g., Simmons, or Headhunter, etc.) to access into the proximal common carotid artery. A relatively stiff (exchange length) guidewire is then placed through this 5 FR or 6 FR diagnostic catheter. The guidewire is then advanced through the common carotid artery, and distally into the external carotid artery to “anchor” this wire. Once the stiff guidewire is in place, it allows the exchange over this wire of a guiding catheter or a long carotid sheath. For the purposes of this specification, we will refer to this guiding catheter or long carotid sheath merely as a “sheath” or a “carotid sheath.”

The present invention is a thin-walled, flat wire reinforced sheath with a differential in sheath flexibility from the proximal portion to the distal portion of the sheath. Specifically, the carotid sheath described herein would have a greater stiffness along most of its proximal length and more flexibility to enhance sheath tracking over a guidewire, a diagnostic catheter, or dilator in the distal portion of the sheath. At this time, there is no sheath that exists in the world that has a comparatively long (about 80 cm) proximal portion that is quite stiff to provide the needed pushability for a long sheath with a comparatively short (about 10 cm) distal portion that is highly flexible to provide ready passage through the highly curved vascular anatomy that must be navigated in order to stent a carotid stenosis. Such a sheath would, for the first time, provide for the interventional cardiologist a design for a carotid sheath that would make stenting of a carotid stenosis a much more successful procedure.

Another important aspect of the present invention is the construction of the tubular shaft of the sheath. Existing sheaths have a wall thickness that is typically greater than 13 mils where 1.0 mil =0.001 inch. By using a flat wire helical coil with a wire thickness of approximately 1 mil to 3 mils, which coil has a very thin coating of plastic placed onto its inner and outer surfaces, it is possible to reduce the wall thickness of the tubular shaft to less than 7 mils and preferably to around 5 mils. Such a novel construction would reduce the outside diameter of the introducer sheath by approximately one French size compared to existing sheaths. Such a reduction in the diameter of the sheath would be advantageous in reducing the risk of bleeding at the groin that sometimes occurs after removal of sheaths having a larger outside diameter. Any method to decrease the requirement for surgical repair and/or a blood transfusion often needed for a major bleeding complication would be highly advantageous for the patient and could significantly decrease the morbidity, mortality and cost associated with catheterization procedures.

The present invention also envisions that the shaft of the sheath could employ a thin-walled, flat wire helical coil to be fabricated from a shape memory alloy such as Nitinol to prevent the possibility of kinking of the tubular shaft of the introducer sheath. Still further the present invention envisions a shaft made from two to four separate helical metal coils, one of a cobalt chromium alloy (e.g.; the alloy L605) to enhance the strength and radiopacity of the shaft and the other coil(s) to be made from stainless steel for cost economy. This novel design would be very advantageous for providing a thin-walled shaft for the sheath that is also radiopaque and reasonably economical to build. It is also envisioned that just using one or more stainless steel and/or cobalt chromium alloy flat wires wound onto an inner Teflon layer and then coated in plastic could be an excellent design. Another novel design aspect is to have a differential in sheath flexibility with greater flexibility in the distal portion by either changing the durometer of the plastic components from the sheath's proximal portion to its distal portion (i.e., higher durometer in proximal rather than distal) and/or changing the winding frequency of the helical coil of flat wire as one moves from proximal to distal, such that the distal portion of the sheath is more flexible and trackable than the proximal portion of the sheath.

One object of this invention is to use thin-walled flat wire within the sheath to decrease the outer diameter of the sheath which decreases the size of the vascular entry hole and potentially reduces access site bleeding complications.

Another object of this invention is to create a carotid sheath that has a differential in sheath flexibility such that the distal portion of the sheath is more flexible than the proximal portion of the sheath which provides greater trackability of the distal sheath into the common carotid artery or any other target vessel requiring access for percutaneous intervention.

Still another object of the invention is to have a carotid sheath that is quite stiff for most of its length to enhance its pushability with a distal portion that is much more flexible to ease its passage into the carotid arteries.

These and other objects and advantages of this invention will become obvious to a person of ordinary skill in this art upon reading the detailed description of this invention including the associated drawings as presented herein.

### BRIEF DESCRIPTION OF THE DRAWINGS

FIG. 1 illustrates the present invention having a proximal portion with a length L2, the proximal portion being quite inflexible and a distal portion having a length L1 that is highly flexible.

FIG. 2 is a cross section of the carotid sheath showing the construction details for its proximal portion.

FIG. 3 is a cross section of a distal portion of the carotid sheath showing a means to increase the sheath's flexibility.

### DETAILED DESCRIPTION OF THE INVENTION

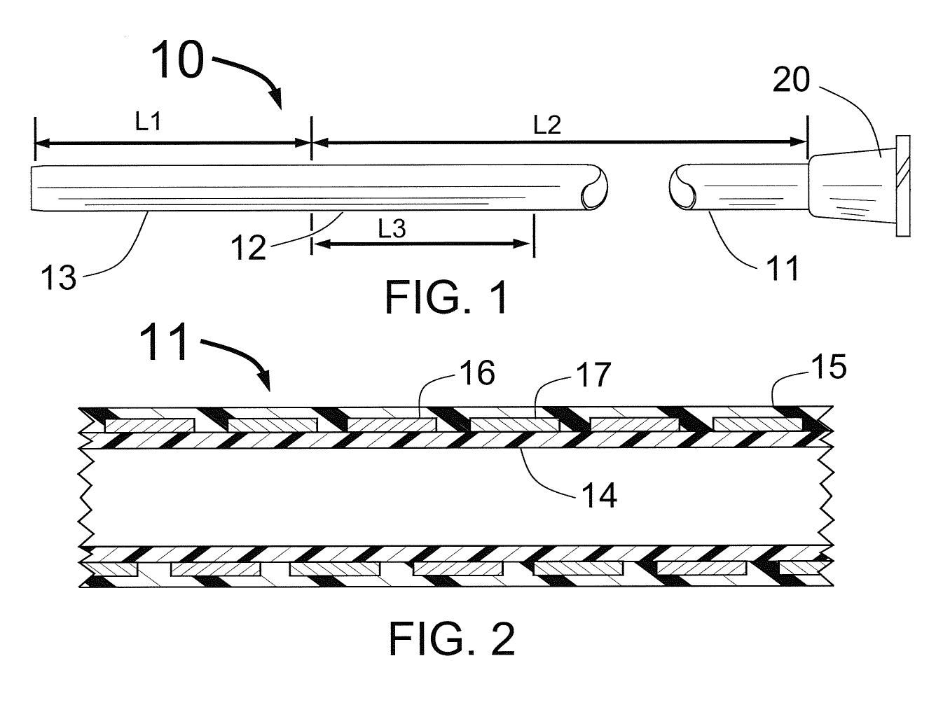

FIG. 1 shows a sheath 10 having a comparatively flexible distal portion 13 having a length L1, a comparatively long and stiff proximal portion having a length L2 and a comparatively short transitional section 12 with length L3. Typical lengths L1, L2 and L3 would be L1=10±5 cm, L2=75±10 cm and L3=3±3 cm. FIG. 1 also shows a Luer fitting at the proximal end of the sheath 10 which is typically used for injecting liquids through the sheath 10 or for connecting a Touhy-Borst fitting for performing carotid stenting. The Luer fitting with the Touhy-Borst fitting also allows for the passage of a dilator. Though it is not shown in FIG. 1, the present invention also envisions having a Touhy-Borst fitting fixedly attached at the sheath's proximal end instead of the Luer fitting.

FIG. 2 shows a typical construction for the proximal portion 11 of the sheath 10. This portion of the sheath tubing would have an interior plastic coating 14 that would typically be formed from a lubricious plastic such as PTFE with a thickness that would typically be less than 1.0 mil, and an exterior plastic coating 15 that would typically be formed from a Nylon type plastic such as Pebax with a thickness between 2 to 5 mils. Either or both coatings could be treated, for example with a hydrophilic coating, to enhance their lubricity.

Between the interior and exterior plastic coatings 14 and 15, an optimum sheath would utilize one or several flat wire helical coils to create tubing that was non-kinking and also radiopaque. At least one helical coil 16 could be formed from a tough, radiopaque metal such as the cobalt chromium alloy L605. At least one additional helical coil 17 would be formed from stainless steel for additional non-kinking resistance and for cost economy. An optimum design might have as many as three separate helical coils of stainless steel and one helical coil of cobalt chromium. It is also conceived to have just one to as many as four helical coils 17 formed from stainless steel. These flat wires would typically have a wall thickness of about 2.0±1.5 mils and a width that could be between 3 and 50 mils. An optimum flat wire would be approximately 2.0 mils thick and about 10 to 20 mils wide. The space between the wires that is occupied by the exterior plastic coating 15 would be approximately 10±5 mils wide for the comparatively stiff proximal portion 11 of the sheath 10. The inside diameter of the sheath would typically be formed to have a small clearance that allows for the passage of catheters that would have diameters between 4 FR and 9 FR. The outside diameter of the sheath would typically be approximately 1.0 FR size greater than the inside diameter of the sheath.

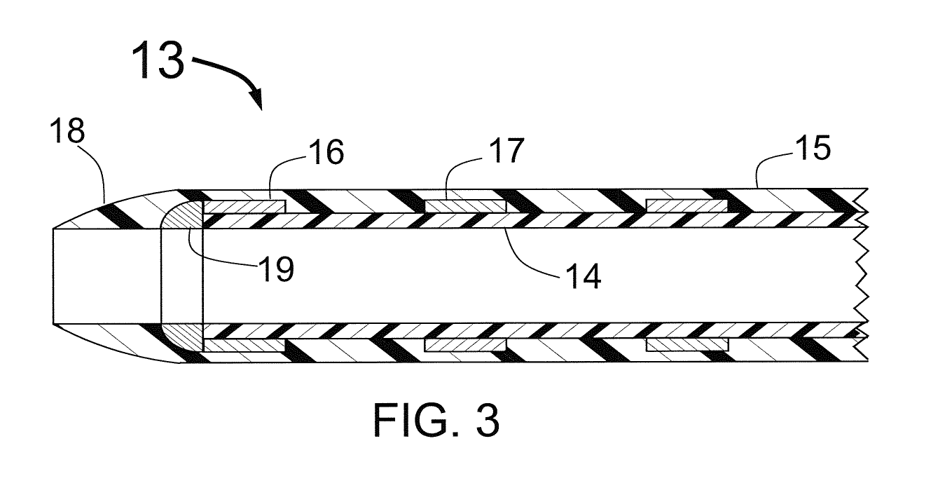

FIG. 3 is a cross section of the distal portion 13 of the sheath 10 showing a tapered radiopaque marker band 19 placed within a tapered tip 18. The distal portion 13 would typically have the same interior plastic coating 14 and exterior plastic coating 15 as is used for the proximal portion 11 of the sheath 10. The increased flexibility of the distal portion 13 can be achieved by a greater separation of the coils of the helical coils 16 and 17 as illustrated in FIG. 3. A greater separation of the flat wire helical coil 16, with the elimination of the helical coil 17 could also be used to provide the desired increased flexibility. Alternatively, the pitch angle of both helical coils 16 and 17 of the proximal portion 11 (as shown in FIG. 3) could be changed to provide increased separation of the coils as another means to provide the increased flexibility that is desired for the distal portion 13. It is certainly envisioned that the sheath 10 would have a single helical coil formed from flat wire with a tight spacing in the proximal portion 11, increased spacing through the transitional section 12 and a comparatively wide spacing of the flat wire helical coil for the sheath's distal portion 13. The separation between the flat wire coils for the distal portion 13 could be as great as 100±90 mils in order to achieve the desired degree of flexibility.

Still another means to improve the flexibility of the distal portion 13 would be to use an exterior plastic coating 15 on that distal portion that has a decreased plastic durometer as compared to a higher durometer that would be used for the exterior plastic coating 15 of the proximal portion 11 of the sheath 10.

What is claimed is:

1. A sheath for accessing a patient's vascular system where most of the length of the sheath is the proximal portion that is distinctly stiffer as to its bending characteristic as compared to a shorter distal portion of the sheath that has increased flexibility.

2. The sheath of claim 1 where the tubing of the sheath has a comparatively lubricious interior plastic coating and an exterior plastic coating formed from a different plastic material and there is at least one flat wire helical coil situated between the two layers of plastic.

3. The sheath of claim 2 where at least one flat wire between the plastic coatings is formed from stainless steel.

4. The sheath of claim 2 where at least one flat wire between the plastic coatings is formed from an alloy of a high density metal such as cobalt chromium.

5. The sheath of claim 2 where at least one of the flat wire helical coils is formed from stainless steel and at least one other helical coil is formed from a metal alloy that is highly radiopaque.

6. The sheath of claim 2 where the increased flexibility of the distal portion of the sheath is achieved by a greater separation between the coils of at least one helical coil of flat wire that is situated between the interior plastic coating and the exterior plastic coating.

7. The sheath of claim 2 where the interior plastic coating is formed from PTFE.

8. The sheath of claim 2 where the exterior plastic coating is formed from Pebax or an equivalent plastic material.

9. The sheath of claim 2 where there is a transitional section of the sheath between the proximal portion and the distal portion, the transitional section having a tighter spacing of at least one flat wire helical coil near the proximal portion of the sheath and a greater separation between the flat wires of the at least one helical coil near the distal portion of the sheath.

10. A sheath for accessing a patient's vascular system, the sheath having a tapered distal end formed from a plastic material with a tapered radiopaque marker band situated within the tapered distal end.

11. A sheath for accessing a patient's vascular system, the sheath having a Touhy-Borst fitting situated at the sheath's proximal end.
